(19) United States
(12) Patent Application Publication
(10) Pub. No.: US 2002/0106692 A1
(43) Pub. Date: Aug. 8, 2002

(54) Screening of target-ligand interactions

(75) Inventors: Richard Ansell, Bedford (GB); Stefan Seeger, Bad Abbach a.d. Donau (DE)

Correspondence Address: SUGHRUE MION, PLLC, 2100 Pennsylvania Avenue, NW, Washington, DC, 20037-3213, US

(73) Assignee: MOLECULAR MACHINES & INDUSTRIES GMBH

(21) Appl. No.: 09/996,571

(22) Filed: Nov. 30, 2001

Related U.S. Application Data
(63) Continuation of application No. PCT/EP00/04948, filed on May 30, 2000.

(30) Foreign Application Priority Data
Jun. 2, 1999 (DE) 199 25 402.8

(51) Int. Cl. G01N 33/53; G01N 33/543

(52) U.S. Cl. 435/7.1; 436/518

(57) ABSTRACT

The invention relates to a method for screening target-ligand interactions while using a chemical library of ligands, to the chemical library of ligands as such, to a method for producing the chemical library, as well as to the use of the chemical library for developing active agents and for developing molecular sensors. The screening method comprises the following steps: (a) measuring at least one fluorescence property of a locally addressable chemical library of ligands that is immobilized on a solid phase, whereby a molecular fluorescence sensor is bound to each ligand; (b) adding the target, and (c) measuring the same fluorescence property/properties of the chemical library as described in step (a). The locally addressable chemical library ligands that is immobilized on a solid phase is characterized in that each ligand is bound to a molecular fluorescence sensor in a preferable manner in which the molecular fluorescence sensor is bound between the ligand and the solid phase and/or to the end of the ligand situated opposite the solid phase.

The invention relates to a method for screening target-ligand interactions while using a chemical library of ligands, to the chemical library of ligands as such, to a method for producing the chemical library, as well as to the use of the chemical library for developing active agents and for developing molecular sensors.

The method of developing new pharmaceutical active agents is complex. After the physiological and clinical aspects of the respective illness have been examined in a first step, the identification of relevant genes and biological target structures, i.e. targets, are as a ride identified for the therapy. The advances in molecular biology and the sequencing methods of DNA and RNA have also offered new possibilities in this identification and thus for the development of pharmaceutical active agents.

With the most recent developments in combinatorial chemistry an advance a the synthesis of ligands for these targets, i.e. molecular structures which interact with the targets, has taken place at the same time as the advances in providing biological targets. Combinatorial chemistry is understood to be the parallel synthesis of a large number of compounds by reaction of known educts in known reactions along the lines of combinatorial principles of automated reaction rules, according to which a large structural plurality of compounds, called chemical libraries, can be produced. Two basic combinatorial principles are known.

Chemical libraries can, for example, be produced by what are called “split-mix” methods, in which microspheres, called beads, are divided up into various reaction vessels, these are again combined after the first synthesis step, e.g. the attachment of the first subsitents or synthesis components, and are divided up again for the second variable substituent. By repeating this process, it is thus possible by a technically simple means to generate thousands or millions of various molecules, with only one specific molecular species being found on each bead. In order to produce a sufficient amount of substance and for a simple handling of the solid phase, porous beads as a rule are used as carrier material.

Another combinatorial principle is what is called the multiple parallel syntheses, in which a certain species is synthesized for each reaction vessel or solid reaction surface. Such chemical libraries are designated as “spatially addressable” libraries. Although the number of synthesizable species is less as compared to the split-mix technology, this method has the advantage, that the identity of the synthesis product is known or can always be verified due to the solid position, and a large amount can be synthesized.

However, it is not the snthesis of the ligands which has proven to be restrictive for the preparation of ligands for the biological targets, but their evaluation, and there is a great need for improved analysis methods for evaluation or for screening of chemical libraries (Burbaum and Sigal, Current Opinion in Chemical Biology, 1997, 1: 72 to 78). For the analysis of target ligand interactions, what are called FACS (Fluorescence Assisted Cell Sorting) methods have been used up to now in particular for libraries according to the “split-mix” method, and in the case of spatially addressable libraries classical assays, such as, for ample, immunoassays in 96-well microtiter plates have been used. In these analysis methods, as a rule, a target binding to a ligand is detected by a fluorescence marker specific for the target Such an analysis method requires, however, rinsing steps before and after the addition of the fluorescence marker as well as a relatively strong interaction between target and ligand.

In view of this background, it is the object according to the invention to provide a method for screening target-ligand interactions in chemical libraries of ligands, which allows a simpler, faster and thus less expensive analysis or evaluation of ligand libraries and which also allows the evaluation of weaker target-ligand interactions.

This object is solved by a method according to the invention for the screening of target-ligand interactions, comprising the steps:

(a) measuring at least one fluorescence property of a spatially addressable chemical library of ligands that is immobilized on a solid phase, whereby a molecular fluorescence sensor is bound to each ligand;

(b) adding the target, and

(c) measuring the same fluorescence property(ies) of the chemical library as described in step (a).

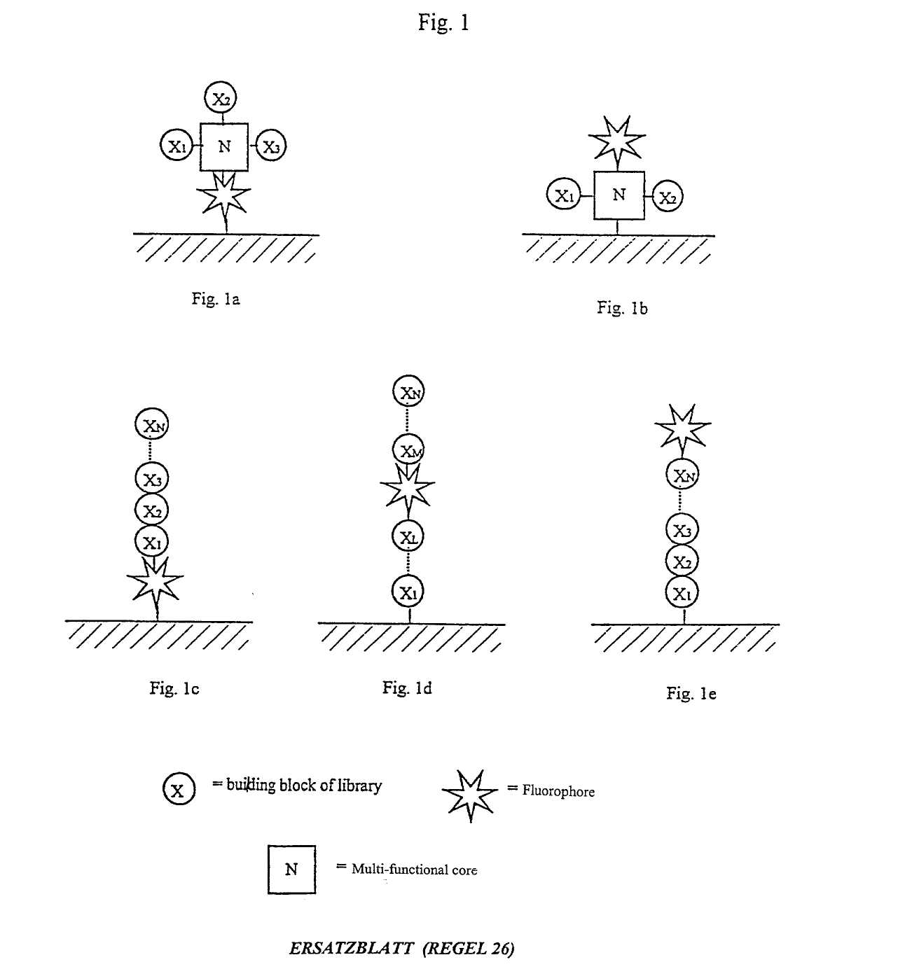

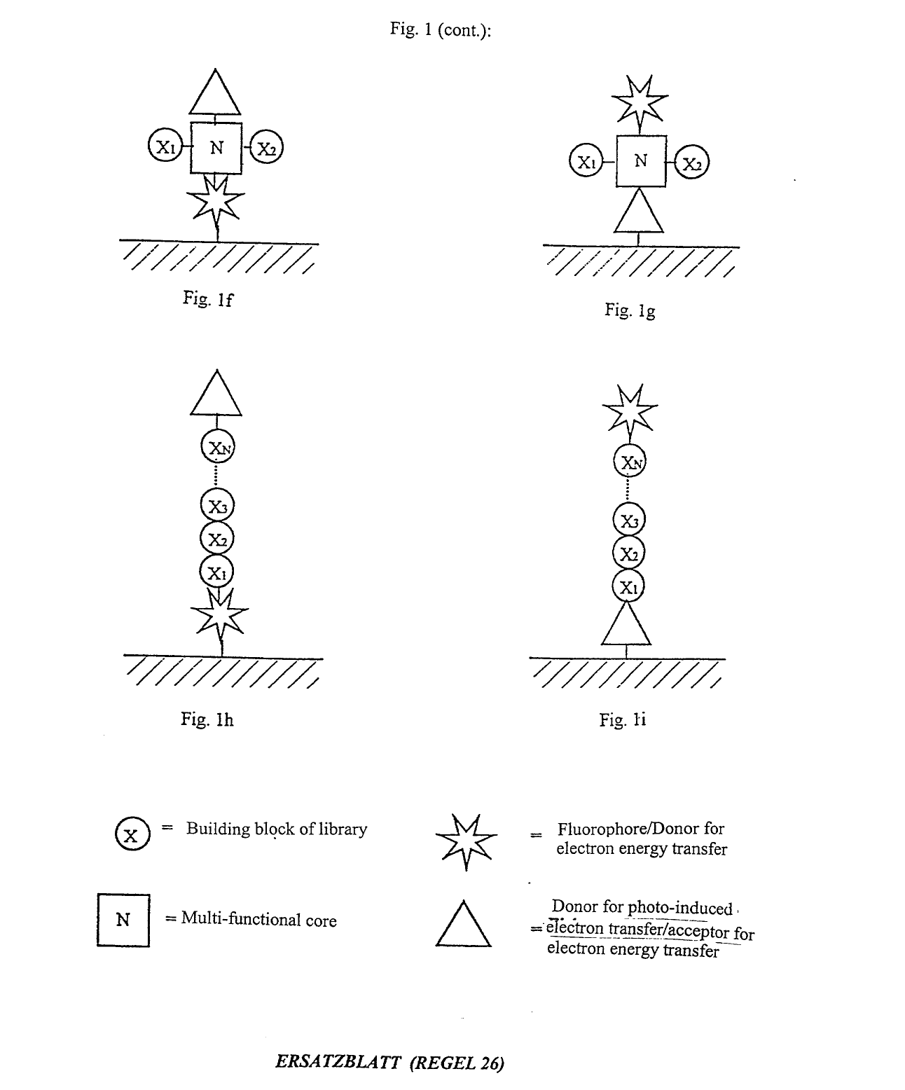

### FIG. 1 shows various possibilities of how the molecular fluorescence sensor can be contained in the ligand.

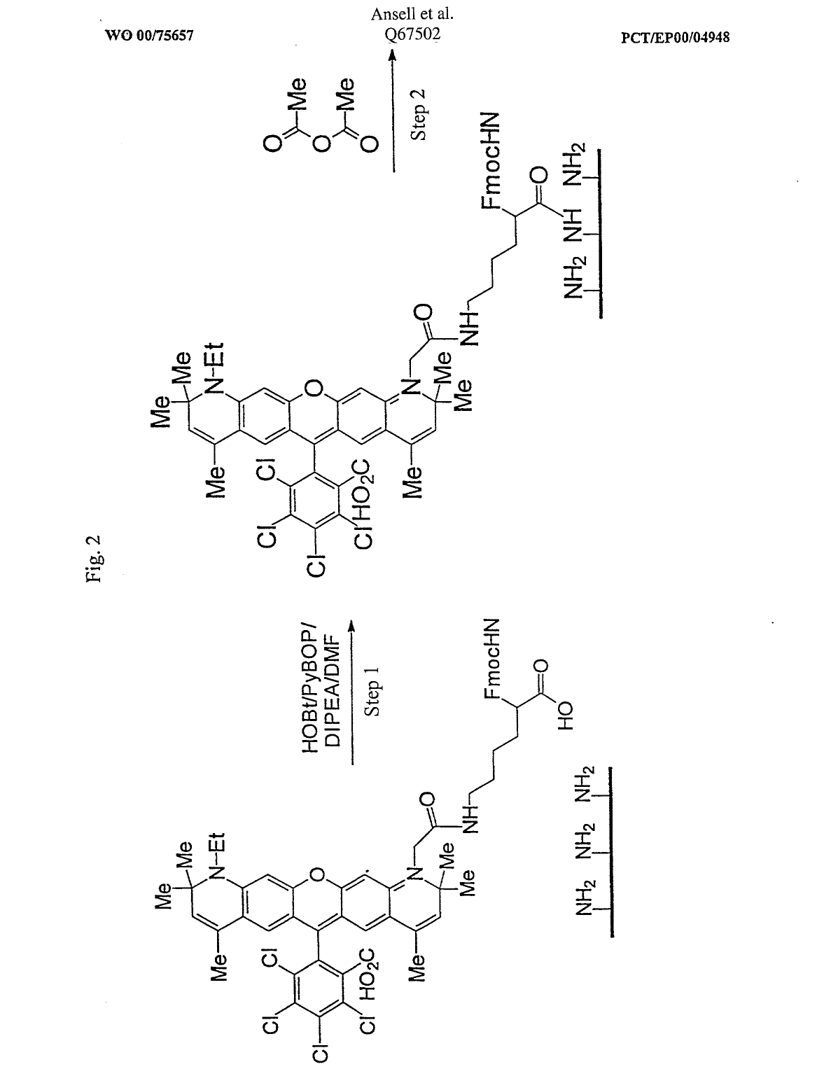

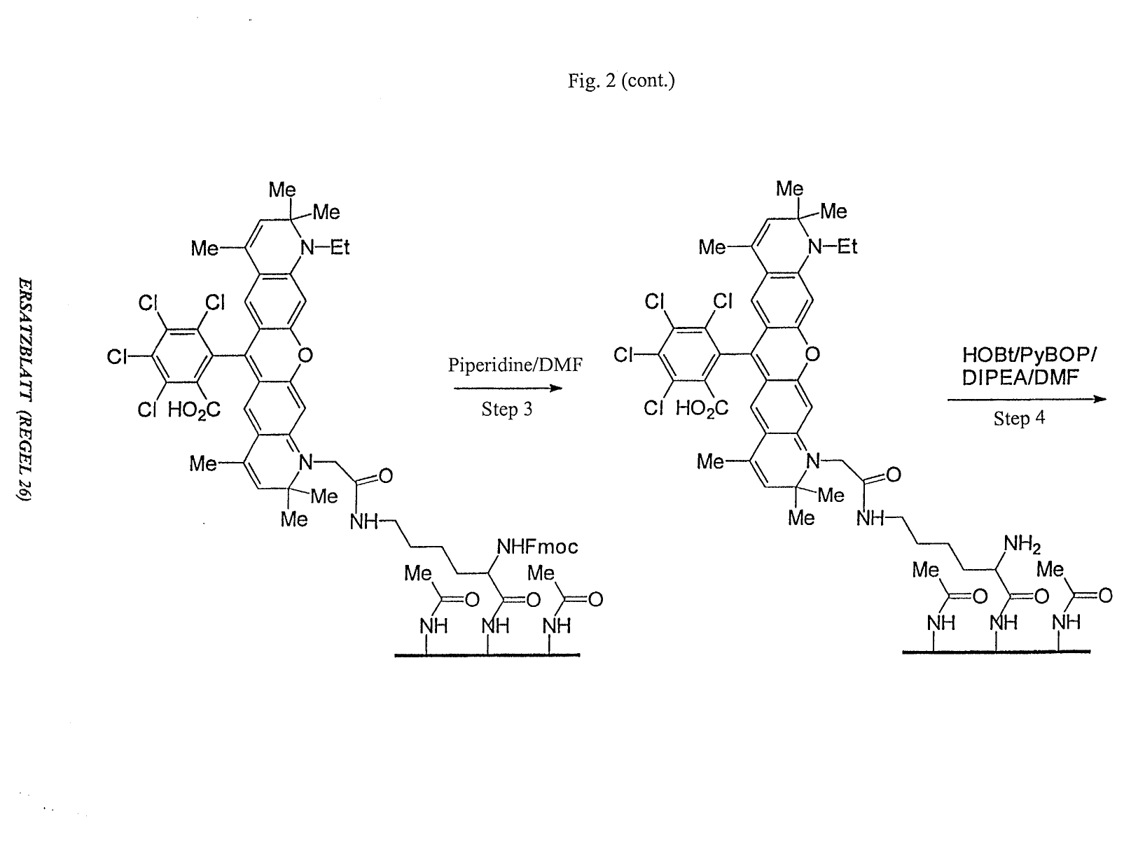

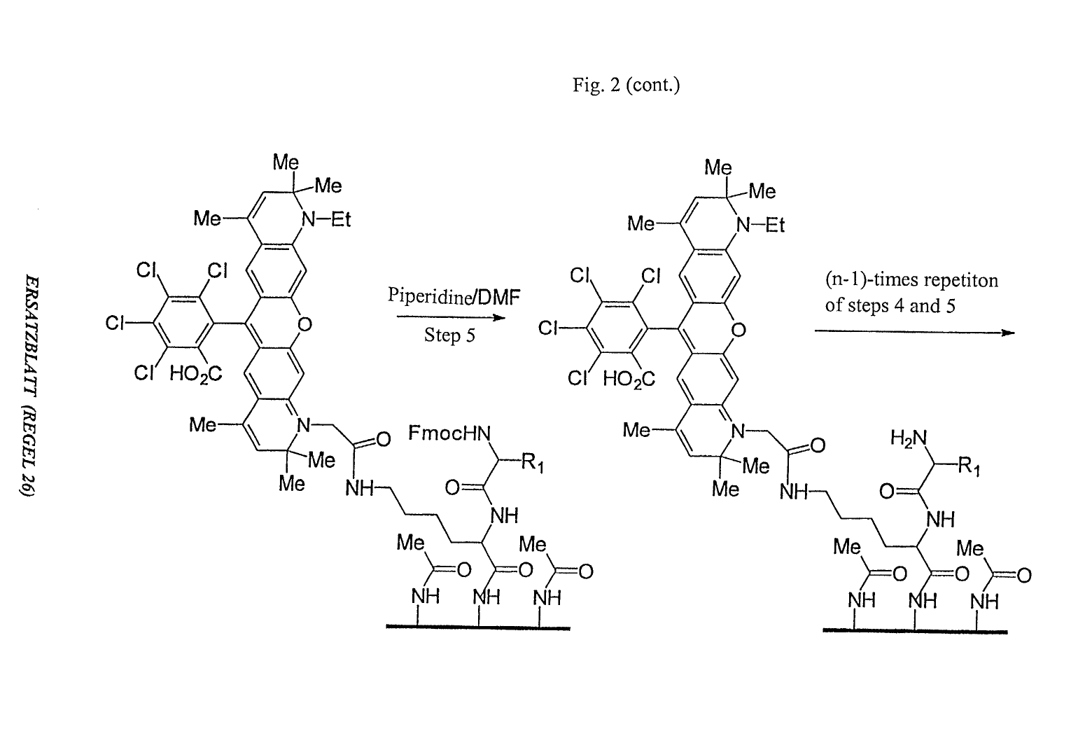

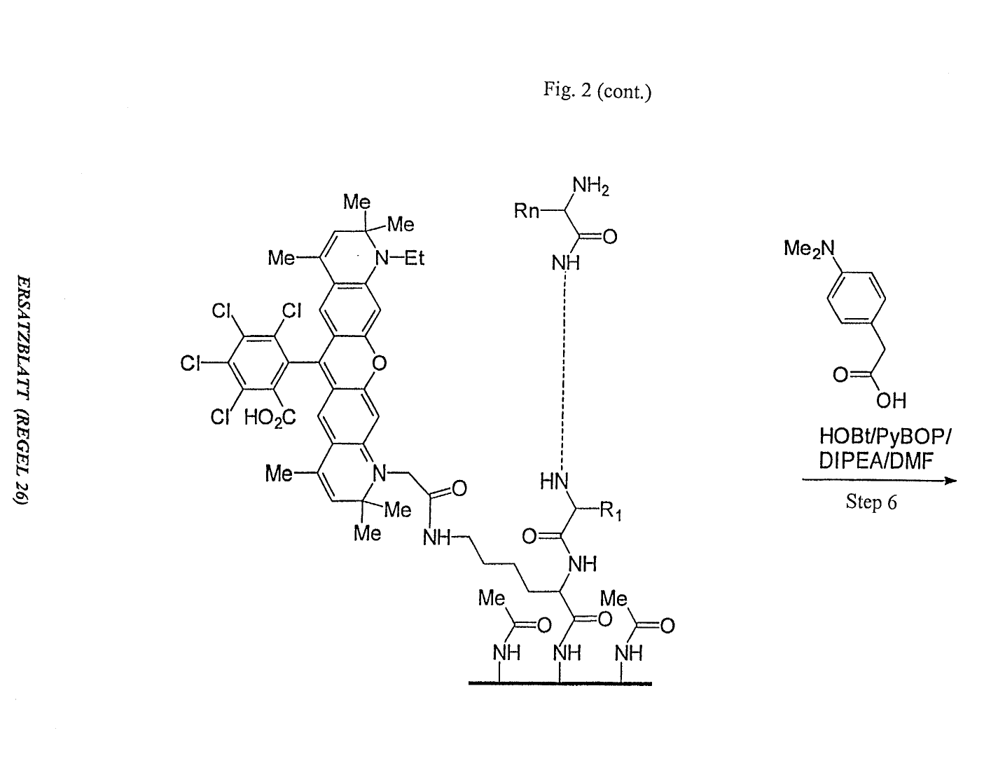

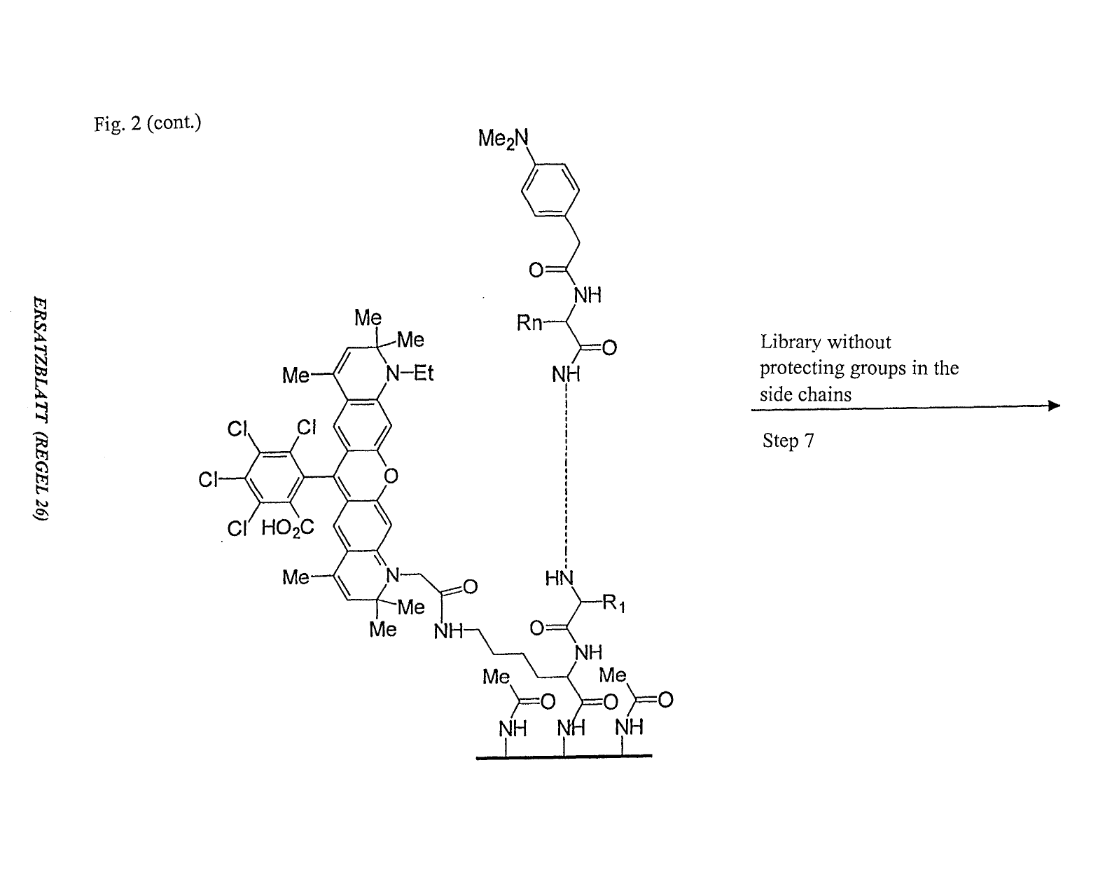

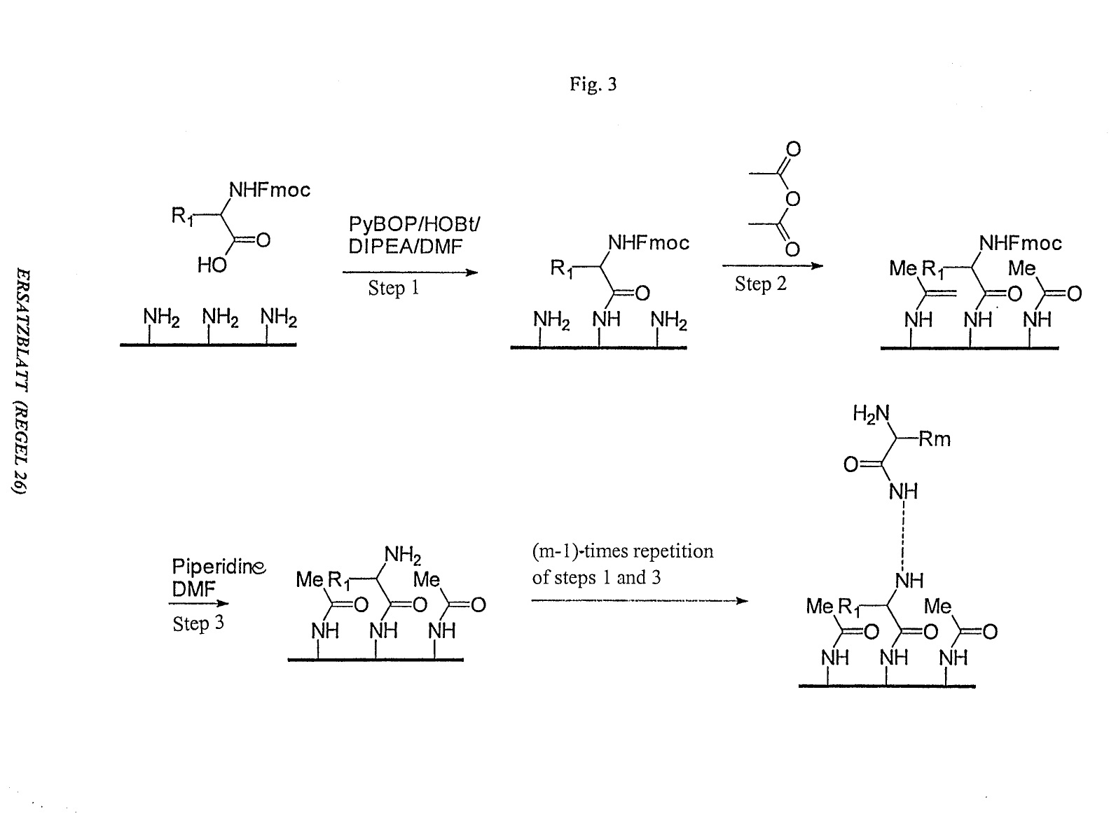

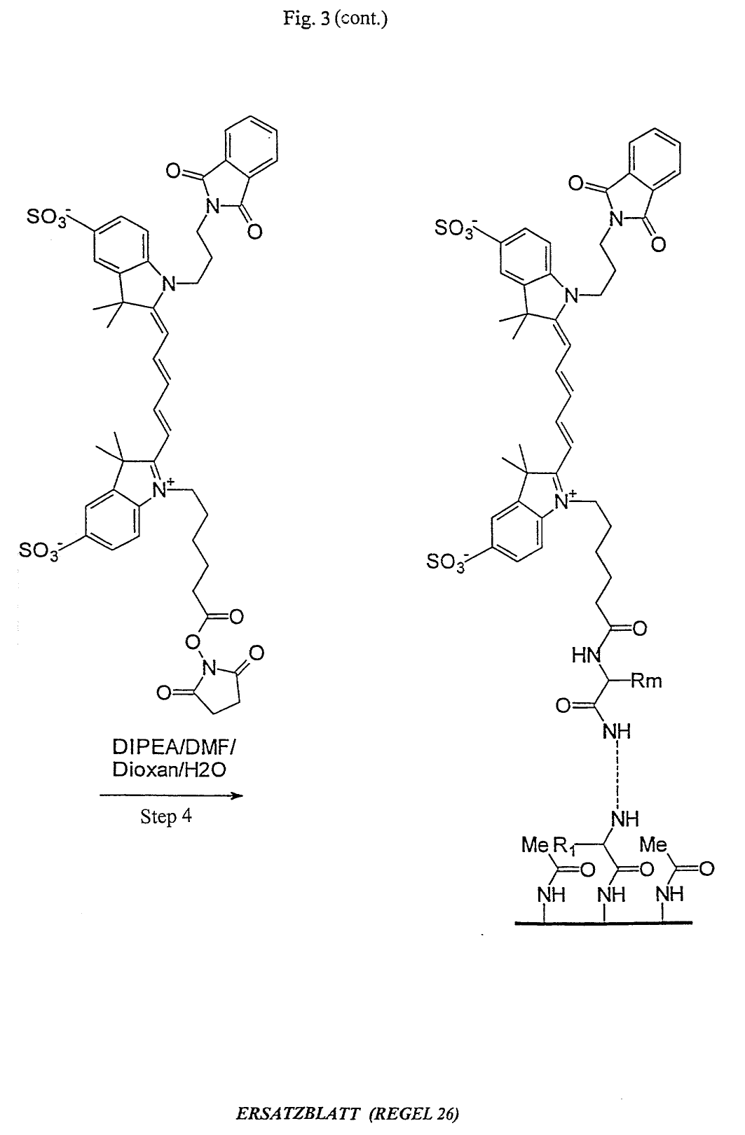

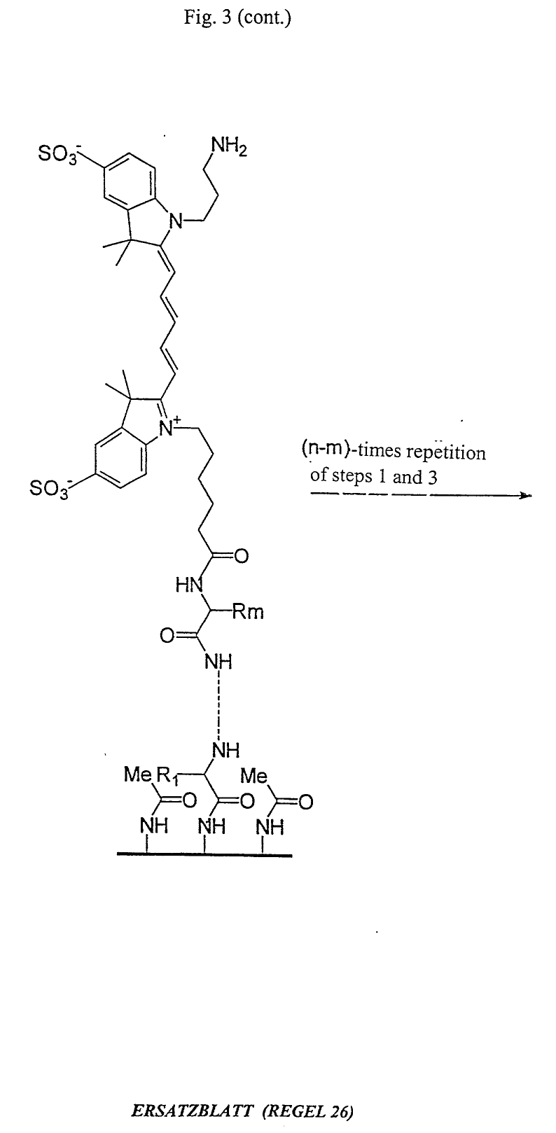

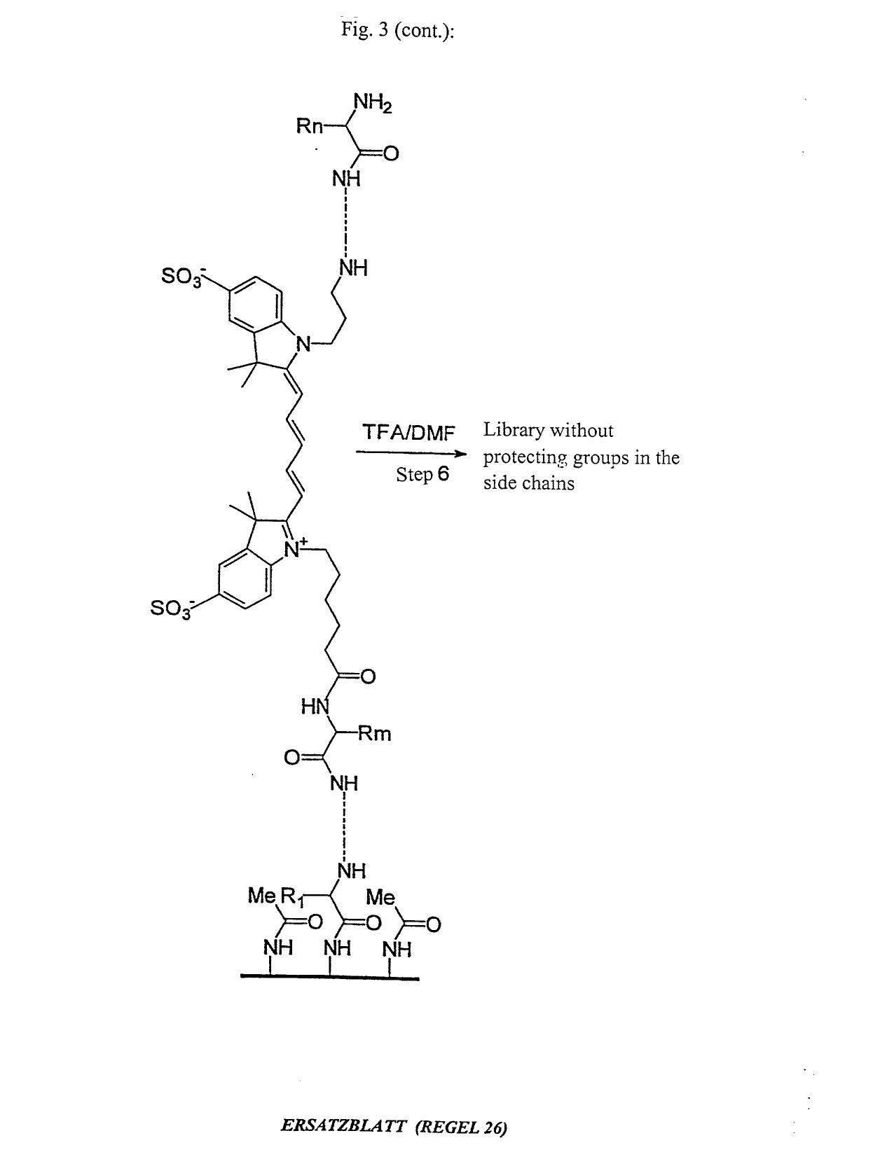

FIGS. 2 and 3 show the diagrams of the reactions on which Examples 1 and 2 are based,

According to the invention the term “target” means molecular target structures for which a molecule is supposed to be found with the screening method which, according to the invention, int s with this target structure.

The term “target” is well-known to the person skilled in the art in particular when searching for new active agents. According to the invention, however, it is not rested to this. When looking for active agents, the targets are, as a rule, identified as the cause for an illness. Regarding this, conventional targets are enzymes, cell surface receptors, nucleus receptors, ion channels and signal transmission proteins or parts of these or also nucleic acids or oligonucleotides.

According to the invention, the term “target”, however, also comprises such target structures for which molecules are supposed to be found which interact with the target in such a manner that this interaction can be used for an analysis of the target. Such molecules are, for example, molecules whose fluorescence properties change due to the interaction with the target. Of special analytical interest here are targets which are of significance in analyses in the medical, environmental and military fields, such as glucose, 2,4-dichlorophenoxy acetic acid and trinitrotoluene, however also larger structures, such as proteins and microorganisms.

The term “ligand” pertains according to the invention to compounds which were synthesized so that they interact with these targets. The expression is thus not restricted to compounds which necessarily interact with the target, but comprise only a binding potential. These ligands are chemically not restricted, to the extent that they can be produced by methods of combinatorial chemistry, i,e. by reaction of known educts into known reactions under automated reaction rules, as a rule, to a solid phase surface. In particular, according to the invention polypeptides are considered as ligands, with Fmoc- or tBoc-protected aminoacids being used for their synthesis. Moreover, the libraries according to the invention can also be non-linear libraries which are derived from a multiple-functional core, such as triazine, where its various functional groups are used for the further assembly of the ligands.

A chemical library of ligands is a collection of ligands produced by parallel synthesis, with the steps of production of the respective ligands differing at lean in one educt. The spatially addressable chemical library according to the invention is produced by a multiple parallel method, with each ligand being present in a space definable by the position, i.e. for example on a defined range of a solid phase surface The chemical libraries according to the invention are bound here to a solid phase surface which corresponds. as a rule, to the surface on which the synthesis of the ligands takes place. To produce spatially addressable libraries, polymer-grafted polyethylene pegs (Geysen it al, Proc. Natl. Acad. Sci. USA, 1984, 81: 39998 to 4002), cellulose membranes (Krchnak, et al., Anal. Biochem 1990, 189: 80 to 83) or functionalized glass object carriers (Fodor et al., Science, 1991, 251: 767 to 773) are especially used.

According to the invention, spatially addressable libraries are preferred which are produced in microtiter plates with 96, 384 or 1536 wells, in particular, when the microtiter plates can also be used for the subsequent fluorescence measuring process, . i.e. the production of the library and its evaluation can be performed in a vessel.

The microtiter plates comprise advantageously an optically transparent bottom plate which consists preferably of glass. The bottom plate comprises in addition preferably a coating which carries suitable functional groups for the covalent immobilization of molecules, for example silane films, Langmuir-Blodgett films or hydrogel films, such as, for example, dextran films. The functional groups on this film are not restricted and include, for example, hydroxy, amino, aldehyde and carboxy groups. Suitable protection groups are known to the person skilled in the art The bottom plate with a Langmuir-Blodgett film is preferred, in particular, a two or three-dimensional crosslinkable Langmuir-Blodgett film, with a Langmuir-Blodgett film coated on a cellulose basis being especially preferred. Such Langmuir-Blodgett films on a cellulose basis have the advantage that they comprise a very minor unspecific adsorption, by means of which the sensitivity in the a detection of target-ligand interactions can be increased at this surface.

The molecular fluorescence sensor used in the method according to the invention is a fluorophore which changes one or more fluorescence property when the target is bound to the ligand, such as e.g. the fluorescence intensity or fluorescence lifetime, by means of which a binding of the target to the ligand can be detected.

Molecular fluorescence sensors used according to the invention can be, in particular:

(1) a fluorophore with an excited, intramolecular charge-transfer condition;

(2) a fluorophore whose fluororescence intensity depends on its moveability;

(3) a fluorophore whose fluorescence is quenched by the target;

(4) a pair consisting of a fluorophore and a donor for photo-induced electron transfer, or

(5) a donor fluorophore/acceptor fluorophore electron energy transfer pair.

A fluorophore with an excited, intramolecular charge transfer condition, what is called a ICT fluorophores, comprises fluorescence properties which are dependent on the polarity of the surrounding solution. Thus, a shifting of the emission maximum or the fluorescence lifetime can be observed with an interaction of a ligand-ICT fluorophore conjugate with a target For example, 5-(dimethyl amino)naphthaline-1-sulfonyl(dansyl)chloride may be mentioned as an example of this, which were used coupled to an antibody against human serum albumin Fab fragments for the detection of human serum albumin (Bright et al., Anal. Chem., 1990, 62: 1065 to 1069). When binding the human serum albumin to the Fab fragments, a great increase of the fluorescene is observed due to the changing of the water coordinates on the fluorophore.

Furthermore, fluorophores can also be used as molecular fluorescence sensors used according to the invention, the fluorescence intensity and/or fluorescence lifetime of which depending on the moveability of the fluorophore. Biscyanine dyes are mentioned here as an example, which, for example, loose their moveability during the complexing of sugars and due to this show a higher fluorescence intensity (Takeuchi et al., Tetrahedron 52, 1996, 1195 to 1204).

Furthermore, pairs of a fluorophore and a donor for photo-induced electron transfer (PET donor) can be used as molecular fluorescence sensors. Two effects can be detected with fluorophore-PET donor pairs: On the one hand, the fluorescence properties such as the fluorescence intensity and/or fluorescence lifetime of such a molecular flurorescence sensor depend as a rule on the distance between the PET donor and the fluorophore, with the fluorescence intensity increasing usually with increasing distance. On the other hand, a change of the fluorescence intensity or fluorescence lifetime can also be caused by a change of the microambience of the ligand when bound to the target.

Furthermore, pairs of donor fluorophores and acceptor fluorophores can be used as molecular fluorescence sensors in the method according to the invention, between which an electron energy transfer can take place. When the donor and the acceptor move close to each other, the acceptor/donor emission ratio increases. For example, Lissamin/Fluorescein are mentioned for such a donor/acceptor pair (Godwin and Burg, J. Am. Chem. Soc. 1996, 118: 6514 to 6515).

According to the invention, the use of molecular fluorescence sensors is especially preferred, the fluorescence of which is quenched by the target, by means of which the fluorescence intensity and/or fluorescence lifetime is decreased. Suitable fluoresce sensors cam be ascertained by previous simple tests, in which the flurophore is brought into contact with the target and the fluorescence intensity or fluorescence lifetime is observed. The larger the change of one of these parameters, the more suitable as a rule is the fluorophore as a molecular fluorescence sensor to be used according to the invention.

The molecular fluorescence sensors used according to the invention comprise preferably a maximum emission wave length in the range of more than 600 nm, since these fluorophores can normally be excited with diode lasers.

The incorporation of the molecular fluorescence sensor into the ligands is illustrated in more detail in FIG. 1. The FIGS. 1a to 1e show examples in which an individual fluorophore is incorporated in the ligand. The FIGS. 1f to 1i show examples in which a fluorophore and a donor for photo-induced electron transfer or a donor fluorophore/acceptor fluorophore electron energy transfer pair are incorporated. In FIGS. 1a, 1b, 1f and 1g the ligand is structured starting from a multi-functional core, in the FIGS. 1c, 1d, 1e, 1g and 1i the ligand is straight-chained. The fluorophores can be incorporated into the ligands in the first (1a, 1c) or the last (1b, 1e) step during the ligand synthesis, or in a intermediate step (1d). A fluorophore and a donor for photo-induced electron transfer or a donor fluorophore/accept fluorophore electron energy transfer pair can be incorporated in any combination in the first, last or in an intermediate step (1f to 1i).

The fluorescence property(ies) to be measured depend(s) on the selection of the molecular fluorescence sensor. According to the invention, the measurement with a confocal fluorescence microscope is preferred. The confocal fluorescence microscope allows, depending on the detector used, a very sensitive determination of the fluorescence intensity, the fluorescence lifetime and even under certain circumstances the number of the binding ligands in particular with very large changes of the respective fluorescence properties. Confocal fluorescence microscopy is suited in particular if the ligands are bound to a planar, transparent solid phase such as, for example, an object carrier. When determining the fluorescence intensity, the fluorescence intensities with various emission wave lengths can also be compared with a fixed, exciting wave length. Since the changes of the respective fluorescence properties are perhaps only very minor, it is preferred to use a detector which is as sensitive as possible. The use of a photo diode is. preferred, in particular a single photon counting Avalanche photo diode. A photomultiplexer or a enhanced CCD camera can be used as an alternative. To measure the fluorescence lifetime, a detector is preferably used which works in the time-correlated single photon counting mode (TCSPC mode),

The use of a spatially addressable chemical library immobilized on a solid phase in the screening method of target-ligand interactions according to the invention is in particular of advantage for the reason that it allows the use of highly sensitive analysis methods in that the possible interaction between ligand and target can take place only in a thin layer on the surface of the solid phase. Furthermore, it allows the subjection of the ligands directly after the synthesis to a screening without a cleavage from the surface, the addition of secondary antibodies or further washing steps being necessary.

The invention pertains moreover to the spatially addressable chemical library of ligands as such immobilized on a solid phase which is characterized in that each ligand contains a molecular fluorescence sensor as defined above. Preferably, the molecular fluorescence sensor lies between the ligand and the solid phase and/or to the end of the ligand situated opposite the solid phase and/or in the middle of the ligand. Furthermore, the incorporation of the fluorescence sensor into the middle of the ligand is especially then preferred when the fluorophore comprises an excited, intra-molecular charge-transfer condition or when its fluorescence depends on its moveability. Especially preferred is a chemical library whose solid phase is made available by the bottom of a microtiter plate.

This molecular fluorescence sensor used in the method according to the invention can be added in each reaction step when assembling the chemical library. According to the invention, the incorporation of the fluorescence sensor is preferred before the first coupling of a synthesis component and/or after the coupling of the last synthesis component In the first case the molecular fluorescence sensor must be bi-functional, in the second case mono-functionality is sufficient. When using donor-acceptor or fluorophore donor pairs, it is preferred to bind the pair to the ligand such that its distance is at a maximum. When producing a chemical library preferred according to the invention, whose solid phase is provided by the bottom of a microtiter plate, the bottom of the wells of the microtiter plate is first derivatized for the covalent coupling of the educts necessary for the synthesis of the ligands. Thereafter, the assembly of the chemical library in the microtiter plate, takes place, with a molecular fluorescence sensor as described above being coupled in a reaction step to the ligands to be assembled.

Moreover, the invention provides the use of such a chemical library for the active agent development as well as for the development of molecular sensors. While the structure of the ligand which interacts with the target is relevant for the active agent development without considering the molecular fluorescence sensor, the conjugate of ligand and molecular fluorescence sensor is of significance for the development of molecular sensors, in this conjugate can be used directly for methods for the analysis of the target.

### EXAMPLE 1

### See FIG. 2

A glass surface is coated with 3-amino propyl triethoxy silane. As an alternative, the glass substrate can be coated with a monolayer of derivatized cellulose using the Langmuir-Blodgett technique. The glass surface is then connected physically with an inert plastic (poly propylene) mask with 96 wells (diameter of the wells: 7.0 mm). In each well a solution of fluorophore derivatives Fmoc-lys-JA53, dissolved in DMF, and the coupling agents HOBt/PyBOP and diisopropyl ethyl amine are added. The dye binds to the surface, after which the wells are washed thoroughly with DMF in order to remove not-specifically adsorbed dye bound only physically. Any amino group not reacted on the surface are then blocked off by reaction with acetic acid anhydride. Thereafter, the Fmoc group is cleaved off from the fluorophore by a solution of piperidine in DMF. A peptide library is then produced with standard techniques of combinatorial chemistry using Fmoc-protected amino acids. In the first step, a Fmoc amino acid and a coupling reagent, such as, for example PYBOP with diisopropyl ethyl amine, are incubized in each well in DMF for several hours, with another amino acid being used in each well. The wells are then washed thoroughly with DMF, after which a solution of piperidine in DMF is added to cleave off the Fmoc groups. The wells ale again washed thoroughly with DMF and the cycle with Fmoc amino acids is repeated until peptides of the desired length are synthesized with various amino acids being used again in each well. In the last ship the protecting group of the last amino acid is removed with piperidine, the wells are washed thoroughly with DMF and the PET electron donor 4-dimethyl amino phenyl acetic acid is coupled in a DMF solution using PYBOP with diisopropyl ethyl amine. In the end, the wells are again washed with DMF, and a solution of trifluoro acetic acid is added to the DMF in order to remove the protecting groups of the side chains. The wells are then washed with DMF, methanol, water and thereafter with buffers for the screening method.

The fluorescence intensity of the ligand in each well of the microtiter plate is measured using a confocal fluorescence microscope with a 635 nm diode laser, which is focused onto a surface of 1 μm² and a single photon Avalanche detector, The target is then added and the measurement repeated. The ligand binds to the target if the fluorescence intensity in a well is changed significantly.

### EXAMPLE 2

### See FIG.

A composite microtiter plate comprising a glass bottom coated with a Langmuir-Blodgett film of amino-functionalized cellulose, and a polypropylene mask adhering thereto is produced as in embodiment Example 1. In each well a solution of m Fmoc amino acid (various amino acids in the respective wells) and the coupling means HOBt, PyBOP and DIPEA are added to DMF (step 1 in Diagram 3). AX the coupling, the wells are again washed thoroughly with DMF and methanol. Any unreacted amino groups on the surface are then blocked off by reaction with acetic acid anhydride (step 2). Thereafter, the Fmoc group is cleaved off from the fluorophore by a solution of piperidine in DMF (step 3). The steps 1 and 3 are repeated until a peptide of the length of m amino acid residues is present in each well. A solution of fluorophore derivatives Cy5 (phthal) (COOSu) is then added in each well in DIPEA/DMF/dioxane/water (step 4 in Diagram 3). The fluorophore is incorporated into the peptide chain and thereafter the wells are thoroughly washed with DMF, methanol and water to remove unspecific, physically adsorbed dyes. The phthalimide group is then cleaved off from the fluorophore by use of a methanolic hydrazine solution (step 5), After this, the steps 1 and 3 are repeated for further (n-m) cycles until peptides of the desired entire length of n amino acid residues are obtained, with Cy5 being incorporated between the amino acid residue m and the amino acid residue m+1 each. Finally, the wells are again thoroughly washed with DMF and a solution of trifluoro acetic acid is added to the DMF to remove the protecting groups on the side chains (step 6). The wells are then washed thoroughly with DMF, methanol and water and then with buffers for the screening.

The fluorescence intensity of the ligands in each well of the microtiter plate is measured using a confocal fluorescence microscope with a 635 nm diode laser which is focused on a surface of 1 μm² on the glass surface and a single-photon Avalanche detector. The target is then added and the measurement is repeated. With significant changes of the fluorescence intensity in a well, the ligand binds to the target

1. Method of screening target-ligand interactions, comprising the steps:
(a) measuring at least one fluorescence property of a spatially addressable chemical library of ligands tat is immobilized on a solid phase, whereby a molecular fluorescence sensor is bound to each ligand,
(b) adding the target, and
(c) measuring the same fluorescence property(ies) of the chemical library as described in step (a),

2. A method according to claim 1, characterized in that the molecular fluorescence sensor is selected from
(1) a fluorophore with an excited, intramolecular charge-transfer condition;
(2) a fluorophore whose fluororescence intensity depends on its moveability;
(3) a fluorophore whose flurorescence is quenched in full or for the most part by the target;
(4) a pair consisting of a fluorophore and a donor for photo-induced electron transfer; or
(5) a donor fluorophore/acceptor fluorophore electron energy transfer pair.

3. A method according to claim 2, characterized in that the molecular fluorescence sensor is a fluorophore whose fluorescence is quenched by the target in full or for the most part.

4. A method according to one of the previous claims, characterized in that the measurement of the fluorescence property is a measurement of the fluorescence intensity or the fluorescence lifetime.

5. A method according to claim 4, characterized in that the measurement of the fluorescence property takes place with a con focal fluorescence microscope.

6. A method according to one of the previous claims, characterized in that the measurement takes place in a vessel in which the spatially addressable chemical library has been produced.

7. A method according to one of the previous claims, characterized in that the measurement of the fluorescence property takes place in a microtiter plate.

8. A spatially able chemical library of ligands immobilized on a solid phase, characterized in that each ligand contains a molecular fluorescence sensor.

9. A chemical library of ligands according to claim 8, characterized in that the molecular fluorescence sensor is incorporated between the ligand and the solid phase and/or to the end of the ligand opposite the solid phase and/or in the middle of the ligand.

10. Chemical library according to claim 8 or 9, characterized in that the solid phase is the bottom of a microtiter plate.

11. Method for the production of said chemical library according to claim 10, comprising the steps:
a) derivatization of the bottom surfaces of the wells of a microtiter plate for the covalent immobilization;
b) assembly of a chemical library of ligands in said microtiter plate, with a molecular fluorescence sensor being coupled in a reaction step to the ligand to be assembled.

12. Use of the chemical library according to one of claims 8 to 10 for the development of active agents.

13. Use of the chemical library according to one of claims 8 to 10 for the development of molecular sensors.
